(19) United States
(12) Patent Application Publication
(10) Pub. No.: US 2013/0152946 A1
(43) Pub. Date: Jun. 20, 2013

(54) Surgical Drape

(71) Applicant: James Peter Sosnowski, Cincinnati, OH (US)

(72) Inventor: James Peter Sosnowski, Cincinnati, OH (US)

(21) Appl. No.: 13/720,331

(22) Filed: Dec. 19, 2012

Related U.S. Application Data
(60) Provisional application No. 61/577,921, filed on Dec. 20, 2011.

(51) Int. Cl. A61B 19/10

(52) U.S. Cl. CPC A61B 19/10
USPC 128/852

(57) ABSTRACT

Embodiments described herein comprise a surgical drape that can include a sheet having an upper portion and a lower portion, one or a plurality of pockets can be associated with the lower portion of the sheet, and one or a plurality of instruments, sponges, or the like that can be retained within the pockets. The drape can include one or a plurality of tethers that can be attached at one end to a pocket and at the other end to an instrument or sponge such that the instrument or sponge can be accounted for during medical procedures.

### REFERENCE TO RELATED APPLICATION

The present application claims priority of U.S. provisional application Ser. No. 61/577,921, filed Dec. 20, 2011, and hereby incorporates the same application herein by reference in its entirety.

### TECHNICAL FIELD

This application relates generally to surgical drapes, and more particularly to surgical drapes having pockets configured to retain tethered sponges.

### BACKGROUND

Surgical sponges are used by surgeons to absorb blood and other bodily fluids in an incision. The sponges are then removed by the surgeons and their assistants during the operation and immediately prior to closure. Sponges should not be left in an incision or body cavity as their presence may result in infections or other problems. At times, sponges may become contaminated even before an operation and require immediate disposal. Thus, a sponge count can be necessary both prior to an operation and after the operation is completed to ensure that one or more sponges have not been left within the incision or body cavity. Vaginal deliveries are unique procedures in which no body cavity is opened but still sponges can be left in the vagina resulting in infections and possible long term pain issues. It is often the case that there is not enough time to count sponges before a precipitous delivery, thus increasing the likelihood of a lost sponge. In addition, vaginal procedures often involve the surgeon turning his back to the surgical field to procure instruments.

As a precaution, one or more sponge counters are often responsible for counting sponges before and after an operation or procedure. Nonetheless, sponges can become lost in an incision or in the vagina, making it necessary for the surgeon to reopen the incision or have the patient undergo an x-ray in order to detect the presence of sponge having x-ray sensitive material impregnated therein. Not only are such procedures undesirable, negligence in counting sponges can result in malpractice suits against doctors and hospitals.

Though much emphasis can be placed on keeping track of surgical sponges, providing a sponge count may not always be an easy task. Sponges may come in several different sizes, and at times, may be small and difficult to find. During an operation, soiled sponges may roll up, stick together, fall on the floor, or get lost among bodily fluids. As multiple surgeons and assistants may be handling several packages of sponges as well as different instruments, it may be difficult to maintain constant focus on the incision. Further, as multiple surgeons and assistants may be working on the incision, it may be difficult to track each sponge as it is used.

Surgical drapes may be used by surgeons to keep a surgical area sterile, and several different shapes and types of surgical drapes have been proposed. However, it can be difficult to easily position items on a surgical drape such that the items can avoid falling or becoming contaminated. Thus, there is also a need to improve the management of items on a surgical drape during an operation.

### SUMMARY

Embodiments described herein can include a surgical drape having a sheet with an upper portion and a lower portion, where a first pocket can be associated with the lower portion of the sheet. The first pocket can define a first cavity. The surgical drape can include a first tether having a first end and a second end, where the first end of the first tether can be coupled with the first pocket. The surgical drape can include a first sponge that can be coupled with the second end of the first tether, where the first sponge can be configured to be at least partially retained within the first cavity defined by the first pocket in a first position and where the first sponge can removed from the cavity defined by the first pocket in a second position.

Embodiments described herein can include a surgical drape having a sheet with an upper portion and a lower portion, where a first sponge pocket can be associated with the lower portion of the sheet. The first sponge pocket can define a first sponge cavity. A first sponge tether can have a first end and a second end, where the first end of the first sponge tether can be coupled with the first sponge pocket. The surgical drape can include a first sponge, where the first sponge can be coupled with the second end of the first sponge tether and can be configured to be at least partially retained within the first sponge cavity defined by the first sponge pocket. The surgical drape can include a first instrument pocket that can be associated with the lower portion of the sheet, where the first instrument pocket can define a first instrument cavity. A first instrument tether can have a first end and a second end, where the first end of the first instrument tether can be coupled with the first instrument pocket. The surgical drape can include a first instrument that can be coupled with the second end of the first instrument tether, where the first instrument can be configured to be at least partially retained within the first instrument cavity defined by the first instrument pocket.

### BRIEF DESCRIPTION OF THE DRAWINGS

It is believed that certain embodiments will be better understood from the following description taken in conjunction with the accompanying drawings in which:

FIG. 1 is a front perspective view depicting a surgical drape having a plurality of pockets associated with a plurality of sponges in accordance with one embodiment, where the drape is shown being used with a patient in a delivery setting.

FIG. 2 is a front perspective view of the surgical drape of FIG. 1, where the surgical drape is shown with a tethered sponge removed from an associated pocket.

FIG. 3 is a front perspective view of the surgical drape of FIG. 1, where the surgical drape is shown with a plurality of tethered sponges removed from a plurality of pockets.

FIG. 4 is a front perspective view depicting a portion of the surgical drape of FIG. 1, where a tethered sponge is shown removed from an associated pocket.

FIG. 5 is a front perspective view depicting a surgical drape in accordance with another embodiment, where the surgical drape is shown with a plurality of pockets holding tethered sponges and a surgical instrument, as well as a tethered sponge removed from an associated pocket.

FIG. 6 is a perspective view depicting a surgical drape in accordance with another embodiment, where the surgical drape is shown with a plurality of tethered sponges removed from a plurality of pockets and positioned in a sponge receptacle and a pouch.

FIG. 7 is a side view of the surgical drape of FIG. 6, where the surgical drape is shown with a plurality of tethered sponges removed from a plurality of pockets and positioned in a sponge receptacle and a pouch.

FIG. 8 is a perspective view depicting a sponge in accordance with one embodiment.

FIG. 9 is a perspective view depicting another sponge in accordance with one embodiment, where the sponge is shown in a generally elongated shape.

FIG. 10 is a perspective view depicting yet another sponge in accordance with one embodiment, where the sponge is shown in a generally triangular shape.

FIG. 11 is a perspective view depicting yet another sponge in accordance with one embodiment, where the sponge is shown in a generally spherical shape.

FIG. 12 is a perspective view depicting a tether in accordance with one embodiment, where the tether is shown with alligator clips.

FIG. 13 is a perspective view depicting another tether in accordance with one embodiment, where the tether is shown with generally fixed connections.

FIG. 14 is a perspective view depicting yet another tether in accordance with one embodiment, where the tether is shown with hook and loop fasteners

### DETAILED DESCRIPTION

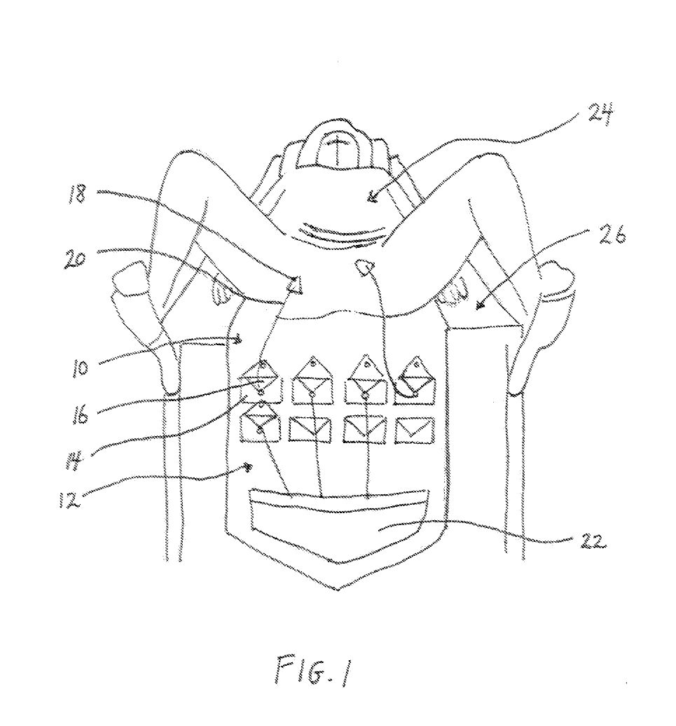

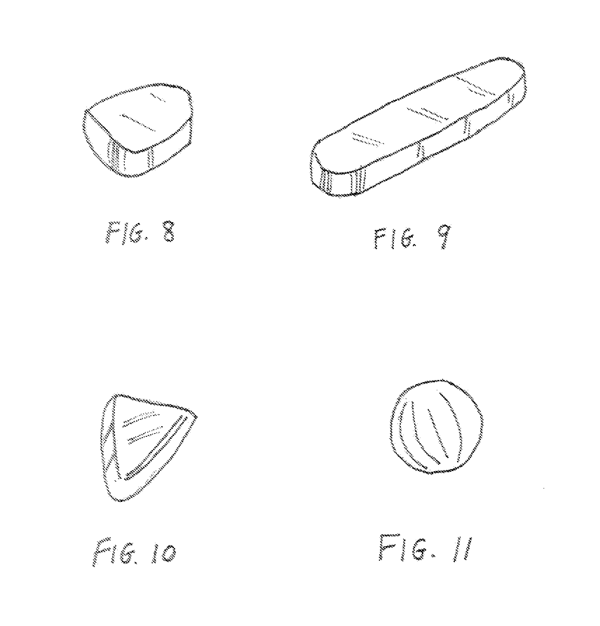

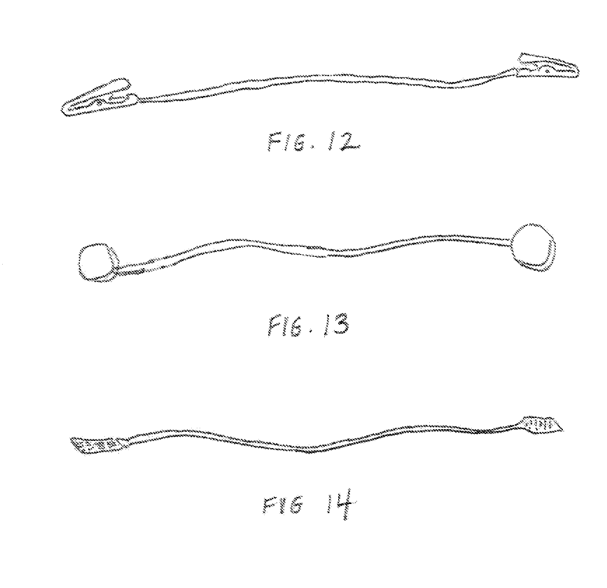

Selected embodiments are hereinafter described in detail in connection with the views and examples of FIGS. 1-14, wherein like numbers indicate the same or corresponding elements throughout the views. FIG. 1 depicts a surgical drape in accordance with one embodiment. It will be appreciated that the surgical drape can take any suitable form, such as a rigid component, a plurality of rigid or semi-rigid components, flexible components, sheets, or the like. Version of the surgical drapes can be formed from materials that are suitable and acceptable for medical use. As shown in FIG. 1, the drape 10 can be oriented on an operating surface 26. FIG. 1 also shows the drape 10 can include a sheet 12, a first pocket 14, a first cavity 16, a first sponge 18, a first tether 20, and a pouch 22. However, it will be appreciated that a drape can comprise a plurality of pockets, cavities, sponges, tethers, and pouches arranged in a variety of suitable configurations. These pockets can be counted and preloaded with the usual instruments or sponges a surgeon or clinician routinely uses and can improve safety, for example, by limiting the number of times sharp instruments are passed from an operative table, obviating the need for the surgeon to take his eyes off the surgical field. These pockets can also assist in reducing contamination, for example, by providing a location to store sponges, instruments, or other items.

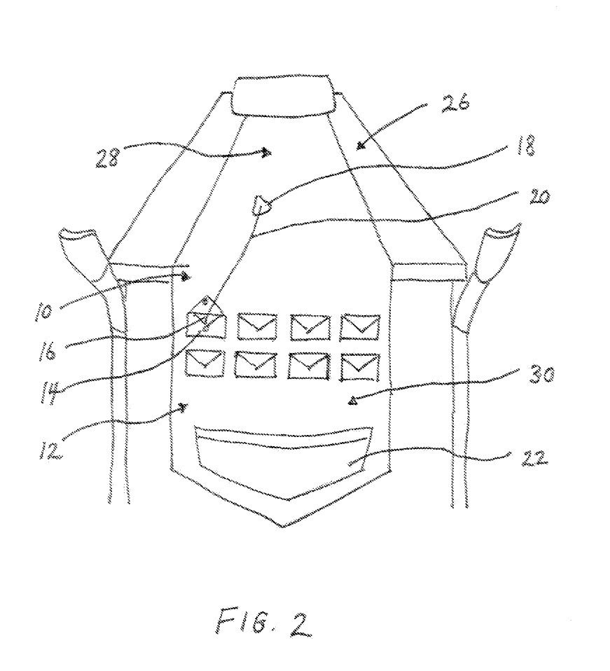

As shown in FIG. 2, the sheet 12 can include an upper portion 28 and a lower portion 30, and the lower portion 30 can extend in a generally downward direction from the upper portion 28. In one embodiment, the lower portion 30 can be generally pivotable with respect to the upper portion 28. As in FIG. 2, the upper portion 28 can be positioned on the operating surface 26, and the first pocket 14 and the pouch 22 can be attached to the lower portion 30. The pouch 22 can be configured to collect fluids originating from a surgical site. In particular, and as shown in FIG. 1, the drape 10 can be positioned with respect to a patient 24 on an operating surface 26 such that the pouch 22 can collect fluids from the surgical site. However, it will be appreciated that a drape can be oriented in any variety of suitable positions. Further, it will be appreciated that a pouch and pockets can be attached to a sheet in any variety of suitable configurations with respect to an upper portion and a lower portion.

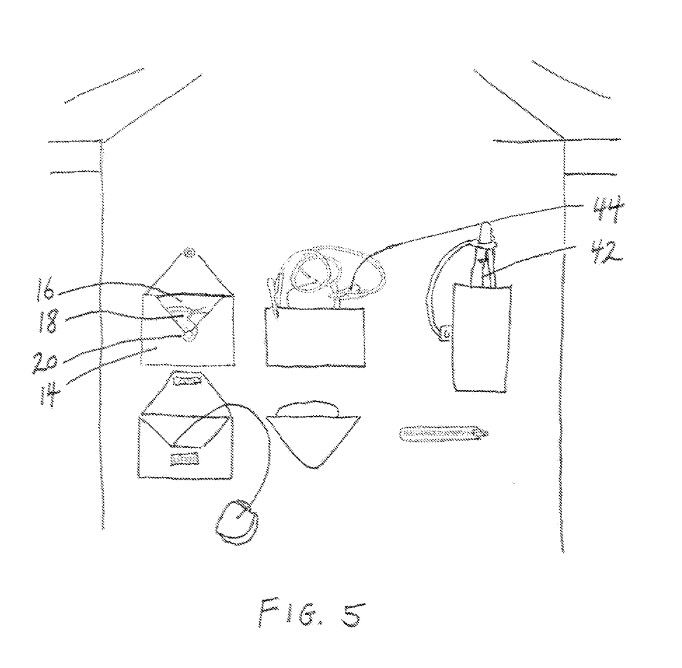

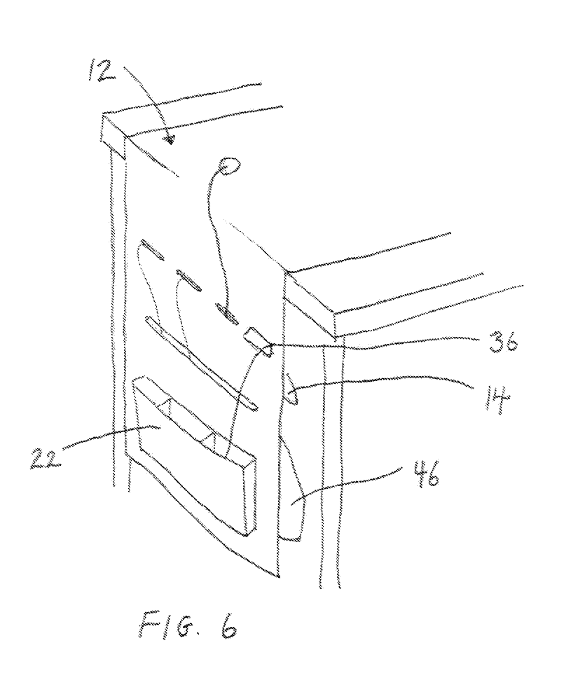

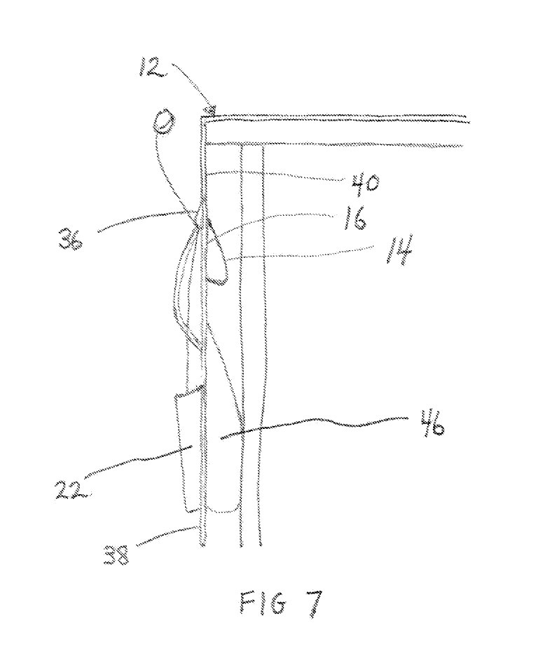

The pouch 22 can have a generally conical shape, as shown in FIGS. 1-3. And as shown in FIGS. 6 and 7, the pouch 22 can be formed to have a generally rectangular shape. However, it will be appreciated that a pouch associated with a sheet can be configured to be a variety of suitable shapes and sizes.

FIGS. 6 and 7 also show that the pouch 22 can be divided into multiple compartments. In one embodiment, the pouch 22 can be graduated to indicate an amount of fluid present within the pouch 22. Further, the pouch 22 can be equipped with a filter that can, for example, be configured to filter out instruments, tissue, or the like of a particular size. It will be appreciated that a plurality of pouches can be attached to a sheet, and that each pouch, or compartment thereof, can be graduated or equipped with a filter.

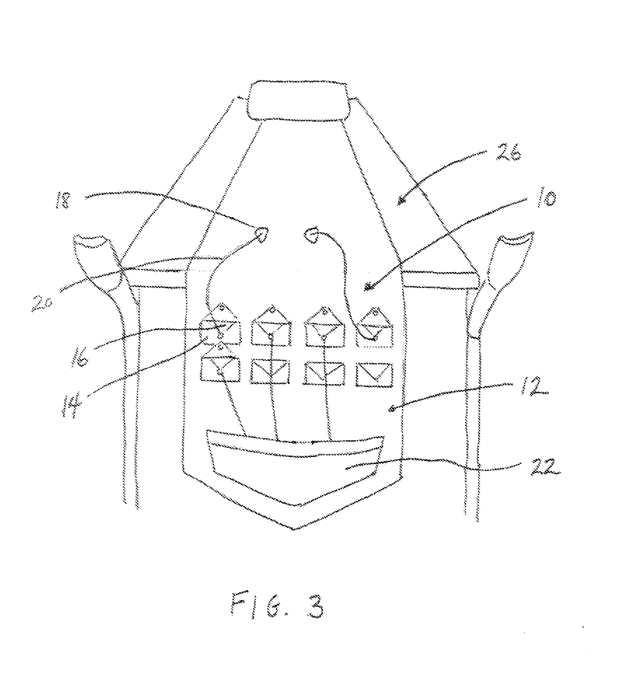

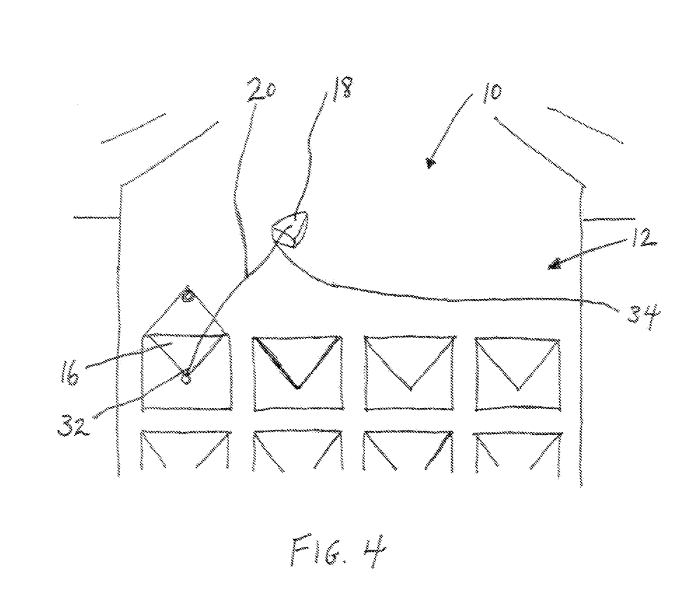

In FIG. 1, the first pocket 14 can define first cavity 16. Also shown in FIG. 1, the first pocket 14 and the first sponge 18 can be coupled by the first tether 20. The first tether 20, as shown in FIG. 4, can have a first end 32 and a second end 34, where the first end 32 can be coupled with the first pocket 14 and the second end 34 can be coupled with the first sponge 18. In one embodiment, the first sponge 18 can be configured for insertion into the drape 10, and as shown in FIG. 5, the first pocket 14 can be configured to receive the first sponge 18 and the first tether 20 within the first cavity 16.

As shown in FIGS. 1-5, the first pocket 14 can be configured to limit access to the first cavity 16 with a snap fastener or any suitable coupling. Each of FIGS. 1-5 shows the snap fastener of the first pocket 14 to be unfastened, such that the first pocket 14 is in an open configuration. The first pocket 14 can be configured to retain the first sponge 18 and first tether 20 within the first cavity 16 in a closed configuration prior to use and, in one embodiment, the first cavity 16 can be a sterile environment. The first pocket 14, in a closed configuration, can be configured to indicate that the first sponge 18 is unused such as, for example, with an unbroken seal or other indicator of use. The first pocket 14 can be configured to limit access to the first cavity 16 with hook and loop fasteners, as shown in FIG. 5, zippers, seals, other suitable fasteners, and combinations thereof. It will be appreciated that pockets can be configured to limit access to corresponding cavities with a variety of suitable fasteners in any suitable combination. The pockets can be configured to project outwardly from the drape, inwardly from the drape, or combinations thereof.

As in FIG. 7, the sheet 12 can include an outer surface 38 and an inner surface 40. As shown in FIGS. 1-5, the first pocket 14 can be attached to the outer surface 38 of the sheet 12. However, as shown in FIGS. 6 and 7, the first pocket 14 can be attached to the inner surface 40 of the sheet 12. It will be appreciated that pockets can be attached in a variety of suitable configurations with respect to an inner surface and an outer surface of a sheet.

In one embodiment, a pocket flap 36 can be configured to cover a portion of the first pocket 14. As shown in FIGS. 6 and 7, the pocket flap 36 can prevent contamination in the first pocket 14. It will be appreciated that a pocket flap can be configured to be a variety of suitable shapes and sizes to cover one or more pockets or any portion thereof.

As shown in FIG. 5, it will be appreciated that pockets associated with a sheet can also be configured to receive and hold an instrument 42 such as forceps, clamps for umbilical cords, or suction bulbs for babies' mouths. In one embodiment, the first pocket 14 can be magnetized to receive needles or instruments. In alternative embodiments, the first pocket 14 can be equipped with a cushion to receive needles or instruments. It will be appreciated that pockets can be configured to be any suitable size for any suitable sponge or instrument. Similarly, as shown in FIGS. 8-11, it will be appreciated that sponges associated with a drape can be configured to be a variety of suitable shapes and sizes or a combination thereof. Further, sponges associated with a drape can be formed with a radiopaque marker. In one embodiment, the sheet can be sold as a kit having a pre-determine number of pockets, sponges, and instruments association therewith where, for example, sheets can include instruments and other materials commonly used for a medical procedure. The inclusion of the instrument commonly used in a procedure may provide an efficient system of providing instrumentation, tracking instrumentation, and counting instrumentation that may not require a clinician to turn their back on a patient.

As shown FIG. 1, the first tether 20 can allow the first sponge 14 to be used in a surgical site, where the presence of the tether 20 indicates that the first sponge 14 is in use and can indicate the general position of the first sponge 14. By connecting the first pocket 14 and the first sponge 18, the first tether 20 can facilitate management of the first sponge 18 throughout a procedure, proper sponge counting at the end of the procedure, and disposal following the procedure. In particular, by locating the first tether 20, the first sponge 18 can be quickly found at any point during the procedure.

In one embodiment, the drape 10 can include a plurality of pockets that can have a corresponding sponge and tether configured for insertion therein. In one embodiment, an accounting of sponges can be performed more quickly and efficiently by associating a number of sponges with an equal number of tethers or pockets such that tethers or empty pockets can be counted to determine the number of sponges in use.

In one embodiment, the first tether 20 can be retractable where, for example the tether can be spring loaded and can be biased to retract, such that the first sponge 18 can easily be returned to the first pocket 14. In another embodiment, the first tether 20 can be elastic to facilitate extension and retraction of the first tether 20. In alternative embodiments, and as shown in FIGS. 6 and 7, a sponge receptacle 46 can be attached to the sheet 22 for collection of discarded sponges.

While FIGS. 1-7 and 13 show tethers with generally fixed connections to pockets and sponges, the first tether 20, as shown in FIGS. 5, 12 and 14, can be selectively coupled with either or both of the first pocket 14 and the first sponge 18, or any other suitable instrument. In particular, FIGS. 5 and 12 show tethers that can include an alligator clip at each of the first end 32 and the second end 34, and as shown in FIG. 14, a tether can have hook and loop fasteners at each of the first end 32 and the second end 34. It will be appreciated that tethers can be configured to have a variety of suitable fasteners, and that a sponge, pocket, or other item to which a tether can be attached can be configured to have a fastener in accordance with a fastener on the tether.

In one embodiment, the first tether 20 can be made from wire, cloth-like materials, elastic materials, or materials such as those used for sutures or fishing line. In one embodiment, the first tether 20 can be made of polyethylene. It will be appreciated that one or a plurality of tethers can be made of a variety of suitable materials.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art.

What is claimed is:

1. A surgical drape comprising:
(a) a sheet having an upper portion and a lower portion;
(b) a first pocket associated with the lower portion of the sheet, wherein the first pocket defines a first cavity;
(c) a first tether having a first end and a second end, wherein the first end of the first tether is coupled with the first pocket; and
(d) a first sponge, the first sponge being coupled with the second end of the first tether, wherein the first sponge is configured to be at least partially retained within the first cavity defined by the first pocket in a first position and wherein the first sponge is removed from the cavity defined by the first pocket in a second position.

2. The surgical drape of claim 1, wherein the lower portion of the sheet further comprises a pouch configured to capture fluids.

3. The surgical drape of claim 2, wherein the pouch further comprises a filter.

4. The surgical drape of claim 1, wherein the lower portion of the sheet further comprises a second pocket, a second tether, and a second sponge.

5. The surgical drape of claim 1, wherein the first pocket further comprises a closure such that the first sponge is completely retained within the first pocket in the first position.

6. The surgical drape of claim 5, wherein the closure comprises an unbroken seal when the first sponge is in the first position.

7. The surgical drape of claim 1, wherein the first tether is formed from an elastic material.

8. The surgical drape of claim 1, wherein the first tether is configured to be retractable.

9. The surgical drape of claim 1, wherein the first tether is selectively removable from the first pocket.

10. The surgical drape of claim 1, where the first sponge is selectively removable from the first tether.

11. A surgical drape comprising:
(a) a sheet having an upper portion and a lower portion;
(b) a first sponge pocket associated with the lower portion of the sheet, wherein the first sponge pocket defines a first sponge cavity;
(c) a first sponge tether having a first end and a second end, wherein the first end of the first sponge tether is coupled with the first sponge pocket;
(d) a first sponge, the first sponge being coupled with the second end of the first sponge tether, wherein the first sponge is configured to be at least partially retained within the first sponge cavity defined by the first sponge pocket;
(e) a first instrument pocket associated with the lower portion of the sheet, wherein the first instrument pocket defines a first instrument cavity;
(f) a first instrument tether having a first end and a second end, wherein the first end of the first instrument tether is coupled with the first instrument pocket; and
(g) a first instrument, the first instrument being coupled with the second end of the first instrument tether, wherein the first instrument is configured to be at least partially retained within the first instrument cavity defined by the first instrument pocket.

12. The surgical drape of claim 11, wherein the lower portion of the sheet further comprises a pouch configured to capture fluids.

13. The surgical drape of claim 12, wherein the pouch further comprises a filter.

14. The surgical drape of claim 1, wherein the lower portion of the sheet further comprises a second sponge pocket, a second sponge tether, and a second sponge.

15. The surgical drape of claim 11, wherein the lower portion of the sheet further comprises a second instrument pocket, a second instrument tether, and a second instrument.

16. The surgical drape of claim 11, wherein the first sponge pocket further comprises a first sponge closure such that the first sponge is completely retained with the first sponge pocket and the first instrument pocket further comprises a first instrument closure such that the first instrument is completely retained within the first instrument pocket.

17. The surgical drape of claim 11, wherein the first sponge tether and the first instrument tether comprise an elastic material.

18. The surgical drape of claim 11, wherein the first sponge tether and the first instrument tether are configured to be retractable.

19. The surgical drape of claim 11, wherein the first sponge tether is selectively removable from the first sponge pocket and the first instrument tether is selectively removable from the first instrument pocket.

20. The surgical drape of claim 11, where the first sponge is selectively removable from the first sponge tether and the first instrument is selectively removable from the first instrument tether.
